(19) United States
(12) Patent Application Publication
(10) Pub. No.: US 2026/0272690 A1
(43) Pub. Date: Sep. 17, 2026

(54) METHOD FOR DEXTEROUS HAND MOVEMENT DECODING BASED ON SYNERGY BASIS MANIFOLD

(71) Applicant: ZHEJIANG UNIVERSITY, Hangzhou (CN)

(72) Inventors: YU QI, HANGZHOU (CN); HUAQIN SUN, HANGZHOU (CN); YUEMING WANG, HANGZHOU (CN)

(21) Appl. No.: 19/318,545

(22) Filed: Sep. 4, 2025

(30) Foreign Application Priority Data
Mar. 12, 2025 (CN) 202510289652X

(51) Int. Cl. A61F 4/00; A61B 5/372; G06F 3/01

(52) U.S. Cl. CPC A61F 4/00; G06F 3/015; A61B 5/372

(57) ABSTRACT

The present invention discloses a method for dexterous hand movement decoding based on synergy basis manifold, including: acquiring electroencephalogram (EEG) signal feature and corresponding hand movement data, performing temporal alignment and preprocessing to obtain a training dataset; constructing a dexterous hand movement decoding model, in the model, the periodic network module maps the EEG signal feature to a phase manifold, obtaining a EEG feature with periodic characteristics; the control network module extracts decoupled spatiotemporal motion control parameters; based on the decoupled spatiotemporal motion control parameters, the time-varying synergy basis decodes to obtain the corresponding hand movements; constructing a composite loss function and training the dexterous hand movement decoding model by using the training dataset; extracting EEG signal feature to be predicted, inputting the EEG signal feature to be predicted into the trained dexterous hand movement decoding model, and decoding corresponding hand movements.

### FIELD OF TECHNOLOGY

The present invention belongs to the field of motor neural signal decoding, and particularly relates to a dexterous hand movement decoding based on synergy basis manifold.

### BACKGROUND TECHNOLOGY

Invasive brain computer interfaces establish direct communication and control pathways between the brain and external devices, demonstrating significant application value in medical fields such as motor function rehabilitation. As the core carrier of human interaction with the outside world, human hands have highly flexible, precise, and almost no need for deliberate control of objects. This delicate hand movement control function plays an irreplaceable role in daily life. Given the fundamental role of hand function in human behavior, achieving continuous and accurate reconstruction of hand movement function has become a key technical challenge in helping disabled populations restore their ability to live independently and perform professionally. Currently, this field has become a key interdisciplinary direction of global research.

For example, the Chinese patent document with the publication number CN113101021A discloses a robotic arm control method based on MI-SSVEP hybrid brain computer interface, which achieves control of the robotic arm through non-invasive EEG data acquisition, online preprocessing of EEG signals, online processing and decoding of EEG signals, generating user intended targets, and translating targets into control instructions and other steps.

At present, the research on brain computer interfaces has progressed to the preliminary stage of achieving dexterous control of robotic arms, that is, 3-4 free high-performance controls have been achieved, enabling autonomous coffee drinking and eating. However, the human hand has 27 degrees of freedom, making it extremely difficult to control the dexterity of the hand, the main challenge lies in the coordination between multiple joints of the hand and nonlinear interactions. The current hand movement decoding work mostly considers the parameters of each joint as independent variables, ignoring the collaborative relationship between joints, resulting in limited effectiveness in hand movement decoding. Therefore, some studies suggest that the brain may regulate high degree of freedom movements in a low dimensional control space (manifold) and mine low dimensional representations from neural signal. Although multiple different manifolds have been discovered in neural signal, the correspondence between these manifolds and hand movement control parameters remains unclear.

On the other hand, although hand movement control has high-dimensional degrees of freedom, these degrees of freedom are not completely independent, but rather redundant. Therefore, some works are based on dimensionality reduction methods to identify low dimensional motion manifolds from complex hand movements, and based on this, decode high-dimensional movements. However, due to the strong dependence of the learning process on assumptions and constraints, different motion manifolds will be obtained under different assumptions, which is obviously inconsistent with the operational control mechanism of the brain. In addition, most studies only consider the motion signal themselves and do not correlate them with brain signals, so it is unknown whether and how the motion manifold and neural signal are related to each other.

Based on the above background, existing research often explores low dimensional hand movement control manifolds separately in the neural signal space or motion space, but often overlooks the connection between the two, which may lead to mismatches between the neural manifold and the motion manifold, thereby limiting the decoding effect of high-dimensional hand motion.

### SUMMARY OF THE INVENTION

The present invention provides a method for dexterous hand movement decoding based on synergy basis manifold, by incorporating both neural signal and motion signal into the joint manifold learning method, the guidance and constraints of EEG data are added to the learning of motion synergy basis, so that the learned synergy basis manifold can better reflect the actual control mechanism of the brain, thereby achieving more accurate and refined hand movement decoding.

A method for dexterous hand movement decoding based on synergy basis manifold, comprising:
(1) acquiring electroencephalogram (EEG) signal feature and corresponding hand movement data, performing temporal alignment and preprocessing to obtain a training dataset;
(2) constructing a dexterous hand movement decoding model based on a synergy basis manifold, comprising: a periodic network module, a control network module and a synergy basis recruitment module;
wherein, the periodic network module is configured to map the EEG signal feature to a phase manifold, obtaining an EEG feature with periodic characteristics; the control network module is configured to extract decoupled spatiotemporal motion control parameters from the EEG feature generated by the periodic network module; based on the decoupled spatiotemporal motion control parameters obtained from the control network module, the synergy basis recruitment module recruits corresponding time-varying synergy basis from a maintained synergy basis dictionary, and the time-varying synergy basis are combined to decode to obtain the corresponding hand movements;
(3) constructing a composite loss function and training the dexterous hand movement decoding model by using the training dataset;
(4) extracting EEG signal feature to be predicted, inputting the EEG signal feature to be predicted into the trained dexterous hand movement decoding model, and decoding the EEG signal feature to obtain corresponding hand movements.

In step (1), extracting spiking band power from an invasive EEG data as the EEG signal feature.

In step (2), the periodic network module is developed based on an autoencoder framework, comprising: a temporal convolutional and a deconvolution layer, a periodic encoding layer and a decoding layer based on Fourier transform, and a phase manifold mapping layer for periodic motion control feature extraction; training of the periodic network module comprises a reconstruction loss for neural signal autoencoding.

In step (2), the control network module comprises two independent feedforward network layers, the two independent feedforward network layers map the EEG feature obtained from the periodic network module into the spatiotemporal motion control parameters containing an amplitude scaling factor and a time offset parameter.

In step (2), the control network module comprises a decoupling module for decoupling the spatiotemporal motion control parameters during the training phase, the decoupling module obtains motion parameters for independent control motion time and space by exchanging parameters between different samples in a batch; training of the control network module comprises a decoupling loss.

In step (2), the synergy basis recruitment module maintains a synergy basis dictionary, the time-varying synergy basis are selected from the synergy basis dictionary based on the amplitude scaling factor and time offset parameter obtained by the control network module; scaling the amplitude by using the corresponding amplitude scaling factor, selecting the time step of the synergy basis by using the time offset parameter, and obtaining the decoding result of hand movement by linearly combining all synergy basis; the training of the synergy basis recruitment module comprises decoding loss of motion data.

In step (2), the synergy basis recruitment module maintains a synergy basis dictionary. During the training phase, the synergy basis dictionary is used as a learnable parameter tensor, the parameters of the synergy basis dictionary and other parts of the network are jointly estimated through backpropagation method.

The parameter learning process of the synergy basis dictionary is constrained by the regularization terms of the dictionary, comprising smooth regularization terms in the time dimension and orthogonal regularization terms between dictionary elements; the training of the synergy basis recruitment module comprises a regularization loss of dictionary learning.

In step (3), the composite loss function comprises reconstruction loss for neural signal autoencoding from the periodic network module, decoupling loss from the control network module, decoding loss from the synergy basis recruitment module, and regularization loss of dictionary learning from the synergy basis recruitment module.

In step (3), during the training phase of the dexterous hand movement decoding model, parameter estimation is performed based on backpropagation method.

Compared with the prior technology, the present invention has the following beneficial effects:
the present invention differs from previous methods of separately constructing neural manifold or motion manifold, the present invention adopts a joint manifold learning method that considers both neural signal and motion signal simultaneously. At the level of neural signal, periodic manifold is used for representation; at the level of motion signal, a synergy basis is introduced to encode multi joint hand movements. At the same time, by associating neural representations with motion representations through motion control parameters, the learning process of hand movement manifold is supervised by neural signal, thereby achieving joint manifold learning of two modal signals. This method makes the hand movement process more in line with the natural control pattern of the brain. The experimental results indicate that the method of the present invention can achieve more accurate and precise decoding of hand movements.

### BRIEF DESCRIPTION OF THE DRAWINGS

FIG. 1 is a flowchart of a framework for decoding dexterous hand movements based on synergy basis manifold in an embodiment of the present invention.

FIG. 2 shows the experimental paradigm diagram of the dataset in the implementation of the present invention.

### DETAILED DESCRIPTION OF THE EMBODIMENTS

The present invention will be further described in detail with reference to the accompanying drawings and embodiments. It should be pointed out that the embodiments described below are intended to facilitate the understanding of the present invention and do not limit it in any way.

All clinical and experimental procedures in the embodiments of the present invention were approved by the Medical Ethics Committee of the Second Affiliated Hospital of Zhejiang University (ethics review number 2019-158, approved on May 22, 2019). The subject was a 74 years old male who suffered a C4 grade cervical injury and was paralyzed in all four limbs due to a car accident. The subject could only move his neck and above, and had normal language communication and task comprehension abilities. Two 96 channel Utah cortical microelectrode arrays (Blackrock Microsystems, Salt Lake City, Utah, USA) were implanted in the left primary motor cortex of the subjects to record neural signal. The subjects underwent brain computer interface training tasks every working day and rest on weekends.

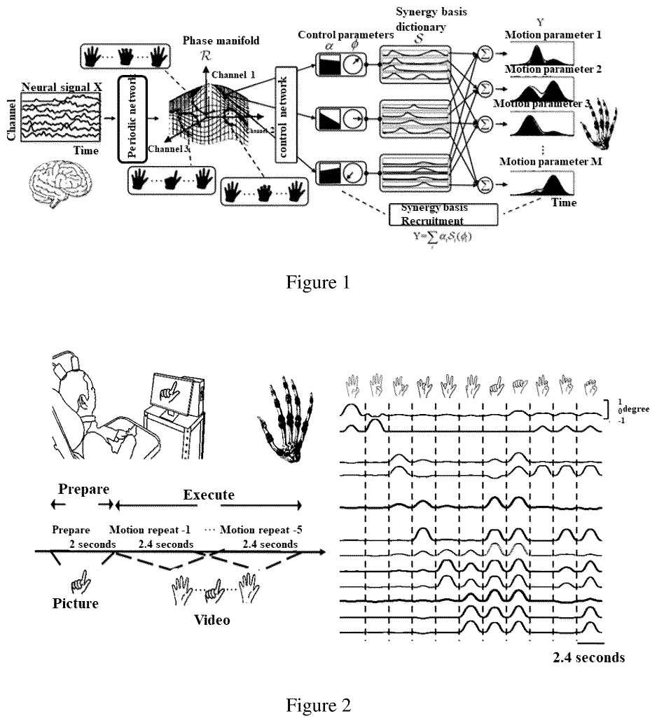

As shown in FIG. 1, a method for dexterous hand movement decoding based on synergy basis manifold, comprising the following steps:

Step 1, data preprocessing: acquiring EEG signal feature and corresponding hand movement data, performing temporal alignment and preprocessing to obtain a training dataset; extracting spiking band power from an invasive EEG data as the EEG signal feature.

The experimental paradigm for data collection is shown in FIG. 2. The dataset included a subject's virtual hand movements while watching instructional videos, as well as the EEG signals and corresponding motion signal collected when imagining performing the same hand movements. Each experimental trial of the paradigm consisted of two stages: “preparation” and “execution”. In the “preparation” stage, the type of action to be executed would be prompted through pictures and voice prompts; subsequently, during the ‘execution’ phase, the virtual hand would perform the action five times in a row, while the subject observe and imagine himself performing the action as well. The paradigm included 11 different hand movements, namely: thumb bending, index finger bending, middle finger bending, ring finger bending, little finger bending, back four fingers bending, back three fingers bending, five fingers grip, three fingers grip, two fingers grip, and thumb adduction. The completion time for each action was 2.4 seconds; each block contained a random sequence of all 11 actions, and each session contained 4 blocks.

During the execution of the action, the angles of 15 key joints in the hand were recorded as motion data with a frequency of 250 Hz. After smoothing, symmetry processing, and upsampling to 1000 Hz, the temporal motion data Y∈R^{M×d}^{ₜ} of 11 different actions were obtained, wherein M represented the feature dimension of the motion signal, specifically M was 15, and dₜ represented the time required to complete a complete action, specifically dₜ was 2400.

At the same time, two 96 channel Utah cortical microelectrode arrays implanted in the subject's cortex were used to synchronously collect neural signal. And the neural signals were amplified, digitized, and sampled at 30 kHz by using the Neuroport system (Blackrock Microsystems). To denoise the neural signal, high pass (0.3 Hz) and low-pass (7.5 kHz) Butterworth filters were used to filter the data. The collection work was conducted on three different experimental days after implantation: 1035 days, 1081 days, and 1105 days.

Based on the original invasive EEG data sampled at 30 kHz, it was down sampled to 1000 Hz after high-frequency filtering at 250 Hz, frequency band filtering at 3000-1000 Hz, and half wave rectification, and the spiking band power (SBP) was obtained as the decoded EEG signal feature X∈ [symbol] ^{C×d}^{ₜ}, wherein C represents the feature dimension of the EEG signal, specifically C was 196, and dₜ represents the time required to complete a complete action, specifically dₜ was 2400.

Preprocessing EEG and motor data by using sliding windows, and the window length and step size can be set according to data characteristics. In this embodiment, the window length was set to 400 ms and the step size was set to 200 ms, averaging the data within the window to obtain 11 step sizes. For each time step of the motion signal, the EEG signals from the first N time steps were used for prediction in this embodiment, N was 5 in this embodiment. According to the principle of k-fold cross validation, using the k-2 fold data as the training set, the 1-fold data as the validation set, and the 1-fold data as the test set, repeating k times. Normalizing the motion data and the neural data based on the mean and variance obtained from the training set in each fold of data. Using the average performance of all validation sets of k-fold as a criterion to select the optimal model, and using the average performance of the k-fold test set as the final performance metric.

In this embodiment, k was 4, and the data was divided into a training set, a validation set, and a test set in a 2:1:1 ratio by using a 4-fold cross validation method.

Step 2, constructing a dexterous hand movement decoding model based on a synergy basis manifold, comprising: a periodic network module, a control network module and a synergy basis recruitment module.

2-1. a periodic network module, the periodic network module is configured to map the EEG signal feature to a phase manifold, obtaining an EEG feature with periodic characteristics;

Specifically, the periodic network module is developed based on an autoencoder framework, comprising: a temporal convolutional and a deconvolution layer, a periodic encoding layer and a decoding layer based on Fourier transform, and a phase manifold mapping layer for periodic motion control feature extraction. Specifically, the calculation method is as follows:

Firstly, extracting hidden representations from neural signal based on the temporal convolutional layer:

$$L - f ( X ) ;$$

among them, X∈ [symbol] R^{C×N} represents the neural signal containing C channels and N time steps, and L∈ [symbol] ^{D×N} represents the implicit representation with dimension D. f represents a one-dimensional temporal convolutional layer, the convolution kernel scale can be set according to the characteristics of the data. In this embodiment, the number of channels was 196, the number of time steps was 5, and the dimension D represented by the number of channels was between 320 and 512, the optimal one was selected with an interval length of 32. Selecting the optimal time dimension size for the one-dimensional temporal convolutional layer from {3, 5}.

Next, the implicit representation with temporal characteristics was modeled by using a periodic sine function, with parameters including amplitude (A), frequency (F), offset (B), and phase offset (P). To estimate these parameters, performing a fast Fourier transform on the implicit representation L to obtain Fourier coefficients c∈ [symbol] ^{D×(J+1)}, where

$$J = ⌊ \frac{N}{2} ⌋ .$$

Then, based on the coefficient sizes of each frequency component, the amplitude Aᵢ, frequency Fᵢ, and offset Bᵢ of each i dimension's hidden representation were calculated through weighted averaging:

$${A}_{i} = \sqrt{\frac{2}{N} {\sum }_{j = 1}^{J} {p}_{i , j}} , {F}_{i} = \frac{{\sum }_{j = 1}^{J} ( {f}_{j} \cdot {p}_{i , j} )}{{\sum }_{j = 1}^{J} {p}_{i , j}} , {B}_{i} = \frac{{c}_{i , 0}}{N} ;$$

wherein,

$${p}_{i , j} = \frac{N}{2} \vert {c}_{i , j} \vert$$

represents the power of the frequency component j, f∈ [symbol] ^{J} is the frequency vector. The phase shift P∈ [symbol] ^{D} was learned through two fully connected layers (FC):

$$( {p}_{x} , {p}_{y} ) = FC ( {L}_{i} ) , {P}_{i} = a tan 2 ( {p}_{y} , {p}_{x} ) ;$$

To enhance the periodic representation characteristics of implicit representations for motion control, a periodic control manifold is defined, and the corresponding representations were mapped onto the manifold R=YΨ(A, P)∈ [symbol] ^{2D}.

$${R}_{2 i - 1} = {A}_{i} \cdot sin ( 2 π \cdot {P}_{i} ) , {R}_{2 i} = {A}_{i} \cdot cos ( 2 π \cdot {P}_{i} ) ;$$

To avoid overfitting by relying only on motion data, we added a decoder to the network to reconstruct the original neural signal. The specific method was to reconstruct the hidden representation based on the learned parameters:

$$\hat{L} = A \cdot sin ( 2 π \cdot ( F \cdot 𝒯 - P ) ) + B ;$$

among them, [symbol] ∈ [symbol] ^{J} represents the time window, then using one-dimensional deconvolution to map back \hat{L} to the original neural signal space:

$$\hat{X} = f ( \hat{L} ) ;$$

The time dimension size of the one-dimensional inverse temporal convolutional layer in this embodiment was consistent with that of the one-dimensional inverse temporal convolutional layer.

Online learning minimized the reconstruction loss function between the original neural signal and the reconstructed neural signal:

$${ℒ}_{rec} = loss ( X , \hat{X} ) ;$$

In this embodiment, Mean Square Error (MSE) was used to measure the loss.

2-2. a control network module, the control network module was configured to extract decoupled spatiotemporal motion control parameters from the EEG feature generated by the periodic network module;

Specifically, the control network module comprises two independent feedforward network layers, the two independent feedforward network layers map the EEG feature obtained from the periodic network module into the spatiotemporal motion control parameters containing an amplitude scaling factor and a time offset parameter. Specifically, the calculation method is as follows:

Using two independent feedforward network (FFN) layers to predict amplitude scaling factor (α∈ [symbol] ^{K}) and time offset (φ∈ [symbol] ^{K}) respectively, where K represents the number of synergy basis. Each FFN consisted of two layers of linear transformations and an activation function ReLU in between. To encourage sparsity of activation, an activation function ReLU was added after the feedforward network layer of the amplitude scaling factor; in order to represent the time offset in a relative time style, we limited the value range of φ to (0,1), so we added a scaled and offset transformed activation function Tanh after the corresponding feedforward network. The calculation method is as follows:

$$α = ReLU ( FFN ( R ) ) , ϕ = 0.5 \cdot ( tanh ( FFN ( R ) ) ) + 0.5 ;$$

In this embodiment, the optimal intermediate layer dimension size of FFN was selected from {196,256}.

In order to encourage the control network module to extract the decoupled motion time and the spatial control parameters, during the training phase, the control network included a parameter decoupling module. This parameter decoupling module strengthened the independence of spatiotemporal control parameters during training through a “data augmentation” strategy. Specifically, the execution of a single continuous action could be decomposed into two parts: action category (what action to do) and action style (how to execute the action). So, the corresponding neural control parameters should also have similar decouplable properties, that is, for an action instance, the scaling factor should remain relatively stable throughout the entire continuous action segment; meanwhile, for movements of different categories but with the same style, the pattern of time offset changes should be basically consistent. In order to achieve these two principles, the module randomly exchanged motion control parameters corresponding to samples with the same action category or stage label in each training batch, obtained new spatiotemporal parameter pairs, and then executed the corresponding synergy recruitment process, introducing corresponding decoding losses to encourage independent control of motion parameters. Specifically, the calculation method is as follows:

During the training phase, for each batch of samples (with a sample size of B), the corresponding α∈ [symbol] ^{B×K} and φ∈ [symbol] ^{B×K} were randomly exchanged among samples with the same action category or motion stage label to obtain the exchanged result â∈ [symbol] ^{B×K} and \hat{φ}∈ [symbol] ^{B×K}, the formula is as follows:

$$\hat{α} = {\prod }_{mot} ( α , {ℒ}^{M} ) , \hat{ϕ} = {\prod }_{stage} ( ϕ , {ℒ}^{P} ) ;$$

among them, Π represents the random permutation operation constrained by the corresponding label, [symbol] represents the label of the motion category, and [symbol] ^{P} represents the label of the motion stage. In this embodiment, the sample size for this batch was set to 32.

Then, for the randomly exchanged sample pairs, the synergy recruitment process was performed again to obtain the corresponding motion outputs [symbol] and [symbol] . Comparing similarity with real motion data and introducing exchange loss:

$${ℒ}_{swap} = loss ( Y , \hat{{Y}_{α}} ) + loss ( Y , \hat{{Y}_{ϕ}} ) = loss ( Y , h ( \hat{α} , ϕ , 𝒮 ) ) + loss ( Y , h ( α , \hat{ϕ} , 𝒮 ) ) ;$$

among them, h(⋅,⋅,⋅) represents the synergy basis recruitment function and [symbol] ∈ [symbol] ^{K×M×T} represents the synergy basis dictionary it maintains. In this embodiment, Mean Square Error (MSE) was used to measure the loss.

2-3. a synergy basis recruitment module, based on the decoupled spatiotemporal motion control parameters obtained from the control network module, the synergy basis recruitment module recruited corresponding time-varying synergy basis from a maintained synergy basis dictionary, and the time-varying synergy basis were combined to decode to obtain the corresponding hand movements.

Specifically, the synergy basis recruitment module maintained a synergy basis dictionary, the time-varying synergy basis were selected from the synergy basis dictionary based on the amplitude scaling factor and time offset parameter obtained by the control network module; scaling the amplitude by using the corresponding amplitude scaling factor, selecting the time step of the synergy basis by using the time offset parameter, and obtaining the decoding result of hand movement by linearly combining all synergy basis; the training of the synergy basis recruitment module comprised a decoding loss of motion data. Specifically, the calculation method is as follows:

The synergy basis recruitment module maintained a synergy dictionary, each synergy basis Sᵢ performed scaling and time offset operations based on dictionary based on the amplitude scaling factor and the time offset parameter, respectively, obtaining the synergy basis that have undergone control parameter transformation. Finally, the contributions of all synergy basis were added to obtain motion data output Ŷ∈ [symbol] ^{M} The corresponding representation of the synergy basis recruitment function could be described in the following form:

$$\hat{Y} = h ( α , ϕ , 𝒮 ) = {\sum }_{i = 1}^{K} {α}_{i} \cdot {S}_{i} ( {ϕ}_{i} ) ;$$

However, the synergy basis was discrete in the time dimension, while the time offset φ was continuous. To address this mismatch and recruit synergy basis in an approximately continuous manner, linear weighted interpolation could be used. Finding the two time steps closest to the time offset and calculating the weights wᵢ=φᵢ−└φᵢ┘ of the two time steps based on the distance. So, the recruitment on the synergy basis time could be written as: Sᵢ(φᵢ)=(1−wᵢ)·S_{i,:,└φ}_{ᵢ}_{┘}+wᵢ·S_{i,:,┌φ}_{ᵢ}_{┐}. Therefore, the corresponding recruitment function could be written as:

$$\hat{Y} = h ( α , ϕ , 𝒮 ) = {\sum }_{i = 1}^{K} {α}_{i} \cdot [ ( 1 - {w}_{i} ) \cdot {S}_{i , : , ⌊ {ϕ}_{i} ⌋} + {w}_{i} \cdot {S}_{i , : , ⌈ {ϕ}_{i} ⌉} ] ;$$

Subsequently, comparing similarity with the real motion data, and regression decoding loss was introduced to supervise the learning of network parameters:

$${ℒ}_{reg} = loss ( \hat{Y} , Y ) ;$$

In this embodiment, Mean Square Error (MSE) was used to measure the loss.

The synergy basis recruitment module maintained a synergy basis dictionary [symbol] ∈ [symbol] ^{K×M×T}, K represents the number of synergy basis, M represents the dimension of motion output, and T represents the number of time steps (reflecting time resolution). During the training phase, the synergy basis dictionary was used as a learnable parameter tensor, the parameters of the synergy basis dictionary and other parts of the network were jointly estimated through backpropagation method. In this embodiment, the number of the synergy basis was set to 15, the dimension of motion output M is set to 15, the time steps were set to 20.

The synergy basis recruitment module maintained a synergy basis dictionary. During the training phase, the dictionary parameter learning process of the synergy basis dictionary was constrained by the regularization terms of the dictionary, comprising smooth regularization terms in the time dimension and orthogonal regularization terms between dictionary elements. Specifically, the calculation method is as follows:

To ensure the smoothness of the synergy basis in the time dimension, a smooth regularization term loss [symbol] ₛₘₒₒₜₕ is defined to penalize excessive differences between adjacent time steps, thereby encouraging smooth transitions in synergy over time:

$${ℒ}_{smooth} = {\sum }_{i = 2}^{T} {( \text{?} ( : , : , i ) - \text{?} ( : , : , i - 1 ) )}^{2} ;$$

Meanwhile, to reduce redundancy between synergy basis and encourage their orthogonality in the feature space, we expanded [symbol] in the spatial dimension and then calculated their Gram matrices G∈ [symbol] ^{K×K}. Defining the orthogonal regularization term loss [symbol] _{divergence} as minimizing the distance between G and the unit matrix [symbol] :

$${\text{?}}_{atom} = \text{?} ( : , : ) ;$$

$$G = {\text{?}}_{atom} {\text{?}}_{atom}^{T} ;$$

$${ℒ}_{divergence} = {‖ G - 𝕀 ‖}_{F}^{2} ;$$

among them, ∥·∥_{F} represents the Frobenius norm.

Step 3, based on the losses obtained from each module, adding up all losses with reasonable weights to obtain the overall loss of the model, which is used for network training.

Specifically, the composite loss function comprises a reconstruction loss for neural signal autoencoding from the periodic network module, a decoupling loss from the control network module, a decoding loss from the synergy basis recruitment module, and a regularization loss of dictionary learning from the synergy basis recruitment module. Specifically, the calculation method was as follows:

$${ℒ}_{total} = λ ( {ℒ}_{r e g} + {ℒ}_{s w a p} ) + ( 1 - λ ) {ℒ}_{r e c} + {β}_{s m o} {ℒ}_{smooth} + {β}_{d i v} {ℒ}_{divergence} ;$$

Among them, λ, βₛₘₒ and β_{div} are hyperparameters used to balance the impact of various losses on the model, which can be manually set. In this embodiment, for λ, performing uniform sampling between 0.85 and 0.95, and the optimal one is selected. For βₛₘₒ, performing uniform distribution sampling based on logarithmic between 0.1 to 500, and selecting the optimal one; for β_{div}, selecting the optimal one based on uniform sampling from 0.1 to 1 as β_{div}.

Step 4, model training and performance evaluation: training the network on the training set data, evaluating the fitting degree of the model by the validation set data, selecting the model with the best regression effect, and finally evaluating the model performance by the test set.

Specifically, based on the embodiment, the main parameter settings for model learning were as follows: batch size was set to 32; using the Adam algorithm to optimize parameters, the learning rate was set to select the optimal model based on its performance in the validation set within a uniform sampling range of 0.001 to 0.01, and the weight decay was set to 1e-4; the maximum learning round was set to 5000, and early stopping method was adopted to alleviate overfitting problem. Specifically, after each round, the R² on the validation set was calculated, and the model with the best performance on the validation set was retained. If R² does not show a 1% improvement in performance within 30 rounds, the training was stopped to obtain the final network parameters.

In order to verify the effectiveness of the method for dexterous hand movement decoding based on synergy basis manifold (SynergyNet) proposed by the present invention, it was compared with the Long Short Term Memory (LSTM) neural network, which is commonly used for invasive brain computer interface decoding tasks and has the best performance. The performance was evaluated by using the commonly used Coefficient of Determination (R²), Correlation Coefficient (CC), and Root Mean Squared Error (RMSE) in regression decoding (see Table 1). From Table 1, it can be seen that SynergyNet had significantly improved performance compared to LSTM: R² increased by 8.72%, CC increased by 3.72%, and RMSE increased by 6.80%, indicating that our method had significant advantages in accurately decoding hand movements.

TABLE 1

| Method | R² | CC | RMSE |
|---|---|---|---|
| LSTM | 0.596 | 0.780 | 0.647 |
| SynergyNet | 0.648 | 0.809 | 0.603 |

The above embodiments have provided detailed explanations of the technical solutions and beneficial effects of the present invention. It should be understood that the above embodiments are only specific examples of the present invention and are not intended to limit the present invention. Any modifications, supplements, or equivalent substitutions made within the scope of the principles of the present invention should be included in the scope of protection of the present invention.

1. A method for dexterous hand movement decoding based on synergy basis manifold, comprising:
(1) acquiring electroencephalogram (EEG) signal feature and corresponding hand movement data, performing temporal alignment and preprocessing to obtain a training dataset;
(2) constructing a dexterous hand movement decoding model based on a synergy basis manifold, comprising: a periodic network module, a control network module and a synergy basis recruitment module;
wherein, the periodic network module is configured to map the EEG signal feature to a phase manifold, obtaining an EEG feature with periodic characteristics; the control network module is configured to extract decoupled spatiotemporal motion control parameters from the EEG feature generated by the periodic network module; based on the decoupled spatiotemporal motion control parameters obtained from the control network module, the synergy basis recruitment module recruits corresponding time-varying synergy basis from a maintained synergy basis dictionary, and the time-varying synergy basis are combined to decode to obtain the corresponding hand movements;
wherein, the periodic network module is developed based on an autoencoder framework, comprising: a temporal convolutional layer and a deconvolution layer, a periodic encoding layer and a decoding layer based on Fourier transform, and a phase manifold mapping layer for periodic motion control feature extraction; a training of the periodic network module comprises a reconstruction loss for neural signal autoencoding;
wherein, the control network module comprises two independent feedforward network layers, the two independent feedforward network layers map the EEG feature obtained from the periodic network module into the spatiotemporal motion control parameters containing an amplitude scaling factor and a time offset parameter;
wherein, the control network module comprises a decoupling module for decoupling the spatiotemporal motion control parameters during a training phase, the decoupling module obtains motion parameters for independent control motion time and space by exchanging parameters between different samples in a batch; a training of the control network module comprises a decoupling loss;
wherein, the synergy basis recruitment module maintains a synergy basis dictionary, the time-varying synergy basis are selected from the synergy basis dictionary based on the amplitude scaling factor and the time offset parameter obtained by the control network module; scaling an amplitude by using the corresponding amplitude scaling factor, selecting a time step of the synergy basis by using the time offset parameter, and obtaining the decoding result of hand movement by linearly combining all synergy basis; a training of the synergy basis recruitment module comprises a decoding loss of motion data;
wherein, the synergy basis recruitment module maintains a synergy basis dictionary, during training phase, the synergy basis dictionary is used as a learnable parameter tensor, the parameters of the synergy basis dictionary and other parts of the dexterous hand movement decoding model are jointly estimated through backpropagation method;
(3) constructing a composite loss function and training the dexterous hand movement decoding model by using the training dataset;
(4) extracting EEG signal feature to be predicted, inputting the EEG signal feature to be predicted into the trained dexterous hand movement decoding model, and decoding to obtain corresponding hand movements.

2. The method for dexterous hand movement decoding based on synergy basis manifold according to claim 1, wherein, in the step (1), extracting spiking band power from an invasive EEG data as the EEG signal feature.

3-7. (canceled)

8. The method for dexterous hand movement decoding based on synergy basis manifold according to claim 1, wherein, the parameter learning process of the synergy basis dictionary is constrained by a regularization terms of the dictionary, comprising smooth regularization terms in the time dimension and orthogonal regularization terms between dictionary elements; the training of the synergy basis recruitment module comprises a regularization loss of dictionary learning.

9. The method for dexterous hand movement decoding based on synergy basis manifold according to claim 1, wherein, in the step (3), the composite loss function comprises a reconstruction loss for neural signal autoencoding from the periodic network module, a decoupling loss from the control network module, a decoding loss from the synergy basis recruitment module, and a regularization loss of dictionary learning from the synergy basis recruitment module.

10. The method for dexterous hand movement decoding based on synergy basis manifold according to claim 1, wherein, in the step (3), during a training phase of the dexterous hand movement decoding model, parameter estimation is performed based on backpropagation method.
